(19) United States
(12) Patent Application Publication
(10) Pub. No.: US 2026/0354353 A1
(43) Pub. Date: Nov. 12, 2026

(54) OPTICAL SENSOR AND PREPARATION METHOD THEREOF, AND METHOD FOR RAPID IDENTIFICATION OF BREEDING EGG SEX USING THE SAME

(71) Applicant: ZHEJIANG UNIVERSITY, Zhejiang (CN)

(72) Inventors: Yingchun FU, Zhejiang (CN); Xiaoyu LIU, Zhejiang (CN); Lan YANG, Zhejiang (CN); Lei YUAN, Zhejiang (CN); Jinming PAN, Zhejiang (CN); Yibin YING, Zhejiang (CN)

(73) Assignee: ZHEJIANG UNIVERSITY, Zhejiang (CN)

(21) Appl. No.: 19/672,701

(22) Filed: May 10, 2026

Related U.S. Application Data
(63) Continuation of application No. PCT/CN2024/144427, filed on Dec. 31, 2024.

(30) Foreign Application Priority Data
Nov. 19, 2024 (CN) 202411652957.4

(51) Int. Cl. A01K 45/00; A01K 43/00; G01N 21/76; G01N 21/78; G01N 31/22; G01N 33/08; G06T 7/90

(52) U.S. Cl. CPC A01K 45/007; A01K 43/00; G01N 21/766; G01N 21/783; G01N 31/223; G01N 33/08; G06T 7/90; G06T 2207/10024; G06T 2207/20081; G06T 2207/20084

(57) ABSTRACT

Provided are an optical sensor, a preparation method thereof, and a method for rapid sex identification of breeding egg utilizing the optical sensor. The optical sensor includes a substrate and at least two sensing units arranged thereon, each sensing unit containing a gas-sensitive material capable of optically responding to gases. The gas-sensitive material is prepared into a solution, ultrasonically dispersed or dissolved, and immobilized on the substrate to form an array-type sensor. The method is based on interactions between the gas-sensitive material and gases emitted from breeding eggs, resulting in changes in optical properties such as light absorption or reflection. The optical signals are collected and analyzed to identify the sex of the breeding eggs. The disclosure enables early-stage, non-destructive sex identification of poultry breeding eggs, overcoming limitations of conventional late-stage and invasive methods.

### CROSS-REFERENCE TO RELATED APPLICATION

This application is a continuation of international PCT application serial no. PCT/CN2024/144427, filed on Dec. 31, 2024, which claims the priority benefit of China application no. 202411652957.4, filed on Nov. 19, 2024. The entirety of each of the above-mentioned patent applications is hereby incorporated by reference herein and made a part of this specification.

### BACKGROUND

### Technical Field

The present disclosure relates to the field of gas detection technology, and specifically relates to an optical sensor for visualized olfactory detection, a preparation method thereof, and a method for rapid identification of the sex of breeding egg utilizing the same.

### Related Art

China is a major producer and consumer of eggs, with a total output reaching 29.38 million metric tons (29,380,000 metric tons), ranking first in the world for many consecutive years. In the course of incubating hatching eggs, the sex of chicks is closely linked to economic returns; the ratio of male to female embryos is approximately 1:1. However, because male chicks do not lay eggs and yield little meat, they lack economic value. Consequently, most male chicks are slaughtered shortly after hatching or macerated for the production of feed. Each year, more than seven billion (7,000,000,000) male chicks worldwide are culled by carbon dioxide asphyxiation or by immersion. This not only increases labor costs and results in substantial economic losses, but also gives rise to issues including the underutilization of high-quality avian egg resources and violations of animal welfare. Furthermore, in the broiler industry, significant differences exist between males and females in growth rate, feed conversion ratio, and meat quality; accordingly, sex-separated rearing facilitates precision management of broilers, reduces resource waste, and enhances production efficiency and the overall competitiveness of the broiler sector. Therefore, it is necessary to conduct sex identification of chicken embryos at an early stage of incubation. This measure will not only enable hatcheries to plan and allocate production in an orderly manner, but is also of significant importance for addressing animal welfare concerns, reducing hatching costs and the expenses associated with the disposal of culled chicks, ensuring the rational utilization of breeding egg resources, and improving the economic efficiency of the poultry-egg industry.

At present, the identification of the sex of embryos in breeding eggs relies primarily upon polymerase chain reaction (PCR) technology. Although this method affords a high degree of accuracy, it typically entails lengthy processing times and cumbersome procedures and requires eggshell-breaching sampling, which, to some extent, adversely affects chick embryo development, rendering it unsuitable for use in industrial incubation of hatching eggs. In hatcheries, sex identification is commonly performed at the chick stage by means of cloacal examination, feather-rate examination, and feather-color examination; however, such manual methods depend largely upon subjective human judgment, are time-consuming and of low accuracy, and cannot be conducted until after the chicks have hatched. In addition, they give rise to two categories of issues, namely, high production costs and ethical controversy. Over the past decade and more, in order to resolve the challenge of non-destructive sex identification during the incubation period of breeding eggs, researchers have principally employed machine vision, spectroscopic techniques, and olfactory (odor-based) methods. Machine-vision detection of breeding eggs has focused mainly on morphological recognition and blood-vessel (blood-line) recognition; however, there remains dispute as to whether a correlation exists between the egg shape index and the sex of the breeding egg, and the accuracy of methods predicated upon blood-line recognition remains to be improved. Spectroscopic methods likewise exhibit some drawbacks, being susceptible to interference arising from individual-egg differences such as eggshell thickness, eggshell color, and the orientation in which the egg is placed. Moreover, most spectroscopic approaches require microinvasive eggshell-breaching sampling, with detection times concentrated in the mid-to-late stages of incubation. With respect to avian egg sex identification and detection, a technological gap currently persists in this field, and non-destructive, in-line detection under production conditions has not yet been realized. Accordingly, the development of new methods capable of early, rapid, and precise identification of information pertaining to breeding eggs constitutes a critical bottleneck urgently requiring breakthrough.

Odors may be indicative of, or reflective of, some essential properties of matter and are unique in their spatiotemporal characteristics. In recent years, there has been a growing body of reports concerning the biochemical information inherent in or conveyed by odors; for example, seagulls are capable of identifying different categories of eggs by olfaction, foxes may identify the species and freshness of bird eggs by scent, and zebras and lizards can ascertain the sex and physiological status of conspecifics through olfactory cues. Some studies have focused on the biochemical information reflected in the odors of avian eggs, including, inter alia, fertilization, development, and parental communication. The results indicate that odor-based detection is feasible; however, the accuracy and stability of such detection remain in need of improvement. At present, in the field of gas detection, optical sensors, by reason of their high sensitivity and selectivity, low cost, and small size, are widely employed for real-time, in situ analysis. The most common optical sensors are predicated upon physical adsorption or chemical interactions between chromophores or fluorophores and analytes, resulting in colorimetric or fluorescence changes. By combining array-based sensing technologies with novel image recognition techniques, it is possible, for any given analyte, to generate composite response spectrums constituting a unique optical “fingerprint” spectrum. However, there presently exists a dearth of research employing optical sensing technologies to effect nondestructive identification of sex at early developmental stages in breeding eggs. This field is in urgent need of further exploration and development.

With the rapid development of the Internet of Things (IoT) and artificial intelligence technologies, the demand for intelligent gas detection has been increasing on a daily basis. In recent years, gas sensors have undergone substantial improvements in multiple respects, including appearance and performance. The successful application of new material fabrication techniques—such as nanotechnology, thin-film, and thick-film processes—has created the conditions necessary for the realization of new functionalities in gas sensors, thereby facilitating their progressive development toward miniaturization, intelligence, portability, and flexible wearability. Meanwhile, when combined with IoT, big data, and deep learning, machine vision technology incorporates the image-feature selection phase into the training process, thereby avoiding issues arising from indistinct image features and the attendant difficulty of feature extraction, while enhancing resistance to noise and enabling the precise detection of the constituent components of gas mixtures in the context of intelligent gas detection.

In sum, the current technology lacks any method or solution employing a gas sensor based on optical techniques for non-destructive, early-stage sex identification of poultry breeding eggs.

### SUMMARY OF DISCLOSURE

In view of deficiencies in the relevant technical field, the present disclosure provides an optical sensor for visualized olfactory detection of volatile gases emitted from poultry breeding eggs, together with a novel method for early and rapid sex identification of such eggs, thereby overcoming numerous shortcomings of existing egg sexing techniques, including late determination windows, damage to the eggs, high testing costs, the need for specialized personnel, cumbersome procedures, prolonged analysis cycles, and the inability to perform visual detection. For the first time, the disclosure applies optical sensing technology to the visualized detection of gases from poultry breeding eggs, converting characteristic gas information into image-based signals, thereby successfully achieving early, non-destructive identification of egg sex and providing a novel non-destructive detection approach for early sex identification of breeding eggs.

Specifically, the present disclosure is achieved through the following technical solutions:

### I. An Optical Sensor for Visualized Olfactory Gas Detection

The optical sensor includes a substrate and at least two sensing units loaded on the substrate, the sensing units include gas-sensitive materials that may react with gas and induce optical signal changes.

The gas-sensitive material is used for adsorbing gas or reacting with gas, causing changes in the optical properties of the material, including changes in light absorption/reflection characteristics, photoluminescence changes, and color changes, thereby detecting gas composition and concentration.

The photoluminescence change is exemplified by a change in fluorescence or in phosphorescence.

In preferred embodiments, the gas-sensitive material includes, without limitation, chromogenic moieties that undergo a colorimetric change in response to a gas, including dyes, nanoporous materials, and gold nanoparticles, or fluorescent and/or phosphorescent moieties that exhibit a luminescence response to a gas, including metal-organic frameworks (MOFs), quantum dots, and carbon dots.

### II. A Method for Preparing an Optical Sensor

The gas-sensitive material is configured into a solution and ultrasonically dispersed or dissolved, then fixed on the substrate through dropping, spraying, or printing methods, and dried to obtain the sensing units. The at least two sensing units are uniformly distributed on the substrate to form an array-type optical sensor.

Specifically, the gas-sensitive material is added to a solvent to prepare a solution.

The solvent is water, ethanol, ethylene glycol monomethyl ether, tetrabutylammonium hydroxide, or p-toluenesulfonic acid.

In preferred embodiments, the substrate includes but is not limited to eggshells, paper-based substrates, polymer substrates (polyethylene terephthalate (PET), polyvinyl chloride (PVC) films, polycarbonate (PC) films, etc.), textile substrates, or water/aerogel, etc.

### III. A Method for Rapid Identification of the Sex of Breeding Eggs Using an Optical Sensor

The method adopts an optical sensor composed of several sensing units placed in the environment near the breeding egg to react, or uses the sensor to react with gas from the collected breeding egg, and then collects the optical signal changes of each of the sensing units before and after placement for processing and analysis to obtain the sex of the breeding egg.

The optical sensor performs visualized olfactory detection on volatile gases from poultry breeding eggs, thereby achieving sex identification of breeding egg. The method may collect visual images/spectral data of optical signal changes from the reaction between the sensing unit and the gas from the breeding egg.

The optical sensor includes the sensing units containing at least two different gas-sensitive materials. The method collects optical signal changes of the sensing units with different gas-sensitive materials and combines them to form a specific spectrum, analyzes and processes the spectrum, and then identifies the sex of the detected breeding egg.

Multiple sensing units with different gas-sensitive materials are arranged on the same substrate, and visual images or spectral images are collected under both conditions before and after the entire substrate reacts with the gas from the breeding egg, and a difference map or a color-difference map of the visual images or spectral images under conditions before and after reacting with the gas from the breeding egg is taken as the spectrum.

When the optical signal change is a color change, natural light or an artificial light source illuminates the gas-sensitive material, and the color change of reflected light is collected by cameras, camcorders, etc. The color-difference map is formed as a spectrum by comparing the images collected before and after.

When the optical signal change is a photoluminescence change, a light source with a specific fixed wavelength is adopted as excitation light to irradiate the gas-sensitive material, where the light source is such as a xenon lamp or laser, which causes the material to emit fluorescence or phosphorescence by affecting a luminous efficiency, a luminous intensity, or a luminous wavelength of the material, and the emission spectrum data or images are collected by a spectrometer or a camera. The spectral image differences before and after excitation are compared to generate the color-difference map or a differential spectrum plot as the spectrum.

When the optical signal change is a light absorption characteristic change, the gas-sensitive material is illuminated through a specific fixed wavelength light source, where the light source refers to covering the ultraviolet to a visible light range, causing the material to exhibit new absorption peaks, disappearance of original absorption peaks, or peak position shifts. A spectrometer is utilized to collect absorption spectral, and the spectral image differences before and after excitation are compared, thereby generating the differential spectrum as the final spectrum.

For optical property changes that are color changes or photoluminescence changes, the collected images are processed in the following manner to obtain spectra: specifically including perspective transformation of images, contour detection, region cropping, color analysis, and color-difference map plotting.

First, a perspective transformation is performed on an image by mapping four designated points in the image—namely, the upper-left, upper-right, lower-right, and lower-left—to a standardized rectangle, thereby effecting perspective correction and obtaining a target image.

Thereafter, image preprocessing is performed to adjust sizes of the image, convert the image to grayscale, and subsequently apply Gaussian filtering to reduce noise.

Thereafter, binarization is performed on the image using Otsu's thresholding method to extract contours of the image. The contours are ranked by contour area. The contour of the greatest area is selected to be subjected to an approximation operation to obtain a quadrilateral. The quadrilateral represents an optical sensor region within the image.

Subsequently, after the detected contour region is converted into a rectified frontal view by means of the perspective transformation, rectangular markers are drawn in accordance with the locations of the sensing units, and a plurality of region of interest (ROI) are cropped, each ROI including one and only one sensing unit.

Finally, mean values of R, G, and B color components within the ROI are calculated and designated as the R, G, and B values of the sensing unit corresponding to the region. On the basis of the R, G, and B values of the sensing unit, the color-difference map/the differential spectrum is generated, which serves as the spectrum.

The method specifically includes the following steps:
Selecting breeding eggs; subjecting the breeding eggs to cleaning, disinfection, and related pretreatment steps, and introducing the breeding eggs into incubation;
Placing the optical sensor in a breeding egg environment to allow sufficient reaction with the gas from the breeding egg; or, alternatively, collecting the gas from the breeding egg and exposing the collected gas to the optical sensor;
Acquiring, by an optical information acquisition system, optical signals of the optical sensor before and after the foregoing reaction;
Data processing and model construction: performing data preprocessing and feature extraction on the acquired optical signal, constructing a breeding egg sex-identification model, and thereafter training the constructed breeding egg sex-identification model under labeled conditions;
Finally, using the trained breeding egg sex-identification model to process the optical signal acquired, in a scenario under test, by the optical sensor after reaction with the gas from the breeding egg, thereby obtaining an identification result of the sex of the breeding egg.

The optical signal includes a visible light images or a spectral image.

The reaction mode in the foregoing step includes contact and non-contact modes between the optical sensor and the breeding egg.

The contact mode includes, without limitation, spray-coating the gas-sensitive material onto the eggshell such that the gas-sensitive material directly reacts with volatile gases emitted by the breeding egg, or adhering a flexible sensor to the surface of the eggshell so that the flexible sensor reacts with volatile gases emitted by the breeding egg.

The non-contact mode includes, without limitation: placing the sensor and the breeding egg within the same chamber such that the gas-sensitive material does not physically contact the breeding egg but reacts with the gas volatilized or emitted therefrom; or using a suction pump or a gas sampling bag to first collect the gas volatilized or emitted from the breeding egg and subsequently delivering the collected gas to the optical sensor for reaction.

The optical information acquisition system in the steps described herein includes, without limitation, industrial camera acquisition systems, smartphones, scanners, microplate readers, fluorescence spectrophotometers, color-recognition devices, and ultraviolet spectrophotometers, for acquiring optical and/or visual signal data from the sensors.

In the aforesaid step, the breeding egg sex-identification model for binary-classification discrimination is established on the basis of collected optical signals, including images or spectra. The breeding egg sex identification model includes, without limitation, principal component analysis (PCA), convolutional neural networks (CNN), decision trees and random forests, support vector machines (SVM), artificial neural networks (ANN), and the like.

The breeding egg sex identification model is an EfficientNet-B0-CBAM model and is divided into nine sequential stages. The first stage is a backbone network, which employs only convolutional modules and, specifically, is constituted exclusively of convolutional modules. Each convolutional module is formed, in sequence, by a 3×3 convolution operation with a stride of 2, a batch normalization layer, and a Swish activation function. The second through eighth stages adopt a convolutional block attention module (CBAM) network, each stage employing a feature-extraction module CBAM mainly composed of a channel attention module (CAM) and a spatial attention module (SAM). The second stage includes one feature-extraction module CBAM of size 3×3; the third stage includes two feature-extraction modules CBAM of size 3×3; the fourth stage includes two feature-extraction modules CBAM of size 5×5; the fifth stage includes three feature-extraction modules CBAM of size 3×3; the sixth stage includes three feature-extraction modules CBAM of size 5×5; the seventh stage includes four feature-extraction modules CBAM of size 5×5; and the eighth stage includes one feature-extraction module CBAM of size 3×3.

The feature-extraction module CBAM includes the CAM and the SAM, wherein an original feature map F input to the feature-extraction module CBAM is processed by the CAM to obtain a channel feature map (Mc). The channel feature map Mc and the input original feature map F are then subjected to a multiplication to obtain a channel attention map F′. The channel attention map F′ is subsequently processed by the SAM to obtain a spatial feature map Ms. Then, the spatial feature map Ms and the channel attention map F′ are subjected to the multiplication to obtain a spatial attention map F″, which serves as the output of the feature-extraction module CBAM.

The CAM includes a max-pooling layer, an average-pooling layer, and a multilayer perceptron (MLP). The original feature map F is respectively processed by the max-pooling layer and the average-pooling layer and then input into the multilayer perceptron MLP to respectively obtain a first global descriptor (vector1) and a second global descriptor (vector2). The first global descriptor vector1 and the second global descriptor vector2 are summed and subsequently subjected to a convolutional layer processing to obtain the channel feature map Mc. The spatial attention module SAM includes a max-pooling layer and an average-pooling layer. The channel attention map F′ is processed by the max-pooling layer and the average-pooling layer and is concatenated, and the concatenated output is then subjected to the convolutional layer processing to obtain the spatial feature map Ms. Finally, a ninth stage is formed by sequentially connecting, in series, a 1×1 convolution operation, a global average-pooling layer, a fully connected layer, and a Softmax activation function.

Compared with the existing technology, the advantageous effects of the present disclosure are embodied as follows:
1. The optical sensor provided by the present disclosure is characterized by a compact form factor, ease of operation, low cost, and high throughput, thereby reducing reliance upon large-scale detection instruments and specialized personnel, while simultaneously lowering testing costs, and exhibiting broad prospects for application in on-site detection.
2. At present, most detection techniques for the sex identification of breeding eggs are concentrated in the mid-to-late stages of incubation and at the hatching stage. The present disclosure is capable of accurately identifying the sex of poultry breeding eggs at an early stage of incubation, thereby overcoming the shortcomings of existing breeding egg sex identification methods (namely, late-stage identification and destructiveness), and enabling early, non-destructive sex identification of poultry breeding eggs. This substantially reduces the economic costs incurred during the incubation of breeding eggs and avoids both the waste of poultry egg resources and the ethical controversies arising from the culling of male chicks.
3. The optical gas sensor of the present disclosure is configured to generate a specific “fingerprint” spectrum by acquiring the changes in the optical signals of the sensing units of a sensor array before and after contact with different volatile gas molecules, thereby enabling visualized readout of an artificial olfaction system. No specialized instrumentation is required, and qualitative identification may even be performed with the naked eye.
4. The present disclosure provides a feasible, non-destructive method for sex identification and analysis of poultry breeding eggs via volatile gas analysis, thereby overcoming the shortcomings of existing breeding egg sex identification methods that require breaking the eggshell of the breeding egg to collect samples, and reducing the impact on the development of the breeding egg.
5. The present disclosure, for the first time, combines optical sensors with machine learning to achieve early, nondestructive sex identification of breeding eggs. Through the application of deep learning techniques and improvements to the model, image classification and recognition are performed efficiently, thereby enhancing the accuracy and efficiency of breeding egg sex identification. The disclosure has a high practical value and broad application prospects, and provides new opportunities for realizing intelligent and automation of poultry breeding egg sex identification in the future.
6. In view of practical application scenarios, the present disclosure constructs a convolutional neural network model for the rapid identification of the sex of breeding eggs, wherein an attention mechanism is introduced into the feature-extraction portion of the network so that the network simultaneously focuses on feature information in both the channel and spatial sizes, thereby suppressing unimportant feature information, enhancing the extraction of deep image features, and achieving improved convolutional performance.

### BRIEF DESCRIPTION OF THE DRAWINGS

FIG. 1 is a photograph of test tubes containing thirty-six gas-sensitive dyes prepared in accordance with the present disclosure.

FIG. 2 is a schematic diagram of an optical sensor fabricated in accordance with the present disclosure.

FIG. 3 is a schematic diagram of a structure and a detection principle of the present disclosure.

FIG. 4 is a color-difference map of the optical sensor prepared by the present disclosure before and after detecting gas from a breeding egg on day 6 of incubation.

FIG. 5 is a schematic diagram of an EfficientNet-B0-based network structure adopted by a breeding egg sex identification model constructed by the present disclosure.

FIG. 6 is a schematic diagram of a CBAM feature-extraction module structure introduced in this embodiment.

FIG. 7 is a schematic diagram of an overall network topology structure of a breeding egg sex identification model provided in this embodiment.

FIG. 8 is an accuracy result diagram of using two models to distinguish male and female breeding eggs on day 6 of incubation in the present disclosure.

FIG. 9 is a result diagram of predicting sex of a breeding egg according to the present disclosure.

FIG. 10 is a schematic diagram of a breeding egg tattoo sensor prepared by the present disclosure.

FIG. 11 is a schematic diagram of the breeding egg tattoo sensor after image correction according to the present disclosure.

FIG. 12 is a diagram showing classification accuracy results of two models of the breeding egg tattoo sensor of the present disclosure.

FIG. 13 shows a laser-cut paper-based sensor structure designed by the present disclosure.

FIG. 14 is a schematic diagram of a fluorescence array sensor prepared by the present disclosure.

FIG. 15 is a diagram showing the classification accuracy results of two models of the fluorescence array sensor of the present disclosure.

### DESCRIPTION OF THE EMBODIMENTS

The present disclosure is further described below through specific embodiments in conjunction with the accompanying drawings, but the present disclosure is not limited thereto.

An optical sensor specifically implemented includes a substrate and at least one sensing unit loaded on the substrate, where the sensing unit contains a gas-sensitive material that may react with gas and induce optical signal changes.

The gas-sensitive material is provided to adsorb or interact with gas, causing changes in the optical properties of the material, such as changes in light reflection, absorption, photoluminescence characteristics, etc.. These changes macroscopically manifest as optical/visual responses of the sensing unit, such as changes in color, fluorescence, phosphorescence, etc. The fluorescence changes include but are not limited to fluorescence enhancement, fluorescence attenuation, fluorescence quenching, etc.

In preferred embodiments, the gas-sensitive material includes, without limitation, chromogenic moieties that undergo a colorimetric change in response to a gas, including dyes (e.g., pH indicator dyes, redox indicator dyes, solvatochromic dyes, and complexometric indicator dyes), nanoporous materials, and gold nanoparticles; and fluorescent and/or phosphorescent moieties that exhibit a luminescence response to a gas, including metal-organic frameworks (MOFs), quantum dots, and carbon dots.

Due to differences among gas-sensitive materials, the changes in the optical signals of the respective sensing units under normal (ambient) conditions will likewise differ. The optical or visual variation results from external light being incident upon the gas-sensitive material, reflected therefrom, and subsequently acquired by an optical detection device. The external light may include visible light, ultraviolet light, or the like. The optical detection devices include, without limitation, cameras, camcorders, and spectrometers.

In specific embodiments, with respect to fabrication, the gas-sensitive material is formulated into a solution and immobilized on a substrate by means of drop-casting, spraying, or printing to obtain the sensing unit. At least one sensing unit is disposed on the substrate to form an array-type optical sensor.

In preferred embodiments, the substrate includes but is not limited to eggshells, paper-based substrates, polymer substrates (including, for example, polyethersulfone (PES) films, polyethylene terephthalate (PET), polyvinyl chloride (PVC) films, and polycarbonate (PC) films), textile substrates, hydrogels and aerogels, etc.

The shape and size of the optical sensor are not particularly limited and may be designed as required; by way of non-limiting example, a square measuring 3 cm×3 cm.

For different gases, the changes in optical signals of the same sensing unit will differ; for the same gas, the changes in optical signal of different sensing units are likewise not identical.

The method of the present disclosure employs an optical sensor including a plurality of sensing units, which is disposed in an environment proximate to the breeding egg, and may be arranged in intimate contact with the breeding egg, or, alternatively, gases from the breeding egg are collected and brought into reaction with the optical sensor. Changes in optical signals before and after placement or exposure of each sensing unit are acquired and input into a computer for processing and analysis to perform sex identification of the breeding egg, with the identification ultimately displayed on a display.

The optical sensor performs visualized olfactory detection of volatile gases emitted by poultry breeding eggs, thereby enabling sex identification of the breeding eggs. The method may include acquiring images that depict visual changes occurring prior to and subsequent to a reaction between the sensing unit and the gases emitted by the breeding eggs.

The optical sensor includes sensing units that include at least two different gas-sensitive materials, wherein the method includes acquiring variations in optical signals from the sensing units corresponding to the respective gas-sensitive materials, combining the variations to generate a characteristic spectrum, analyzing the spectrum, and thereby performing sex identification of the breeding egg under test.

The plurality of sensing units, each incorporating a respective gas-sensitive material, are arranged on a common substrate, with the sensing units corresponding to different gas-sensitive materials being disposed at different positions. The entire substrate is positioned in proximity to the breeding egg to permit reaction, or is exposed to gas collected from the breeding egg to permit reaction. Visual images or spectral images of the sensors are acquired before and after the reaction, and a difference map or a color-difference map between the visual or spectral images obtained before and after reaction with the breeding egg gas is used as the spectrum.

Specifically, a visual image or a spectral image of the entire substrate is first acquired as a first image; thereafter, following reaction of the substrate with the breeding egg gas, a visual image or a spectral image is acquired as a second image. A difference map or a color-difference map between the first image and the second image is then calculated to serve as the spectrum.

When the variation of the optical signal manifests as a change in color, irradiation of the gas-sensitive material by natural or artificial light results in a change in the color of the reflected light, which may be acquired by a camera, a camcorder, or other imaging apparatus. By comparing images acquired before and after, a color-difference map is generated and serves as the spectrum.

Where the optical signal variation includes a photoluminescence variation, a light source of a specified wavelength (e.g., a xenon lamp or a laser) is employed as excitation to irradiate the gas-sensitive material, thereby affecting the emission efficiency, emission intensity, or emission wavelength of the material and inducing the emission of fluorescence or phosphorescence. The emitted spectral data or images are subsequently acquired by a spectrometer or a camera, differences between pre-excitation and post-excitation spectral images are compared to generate the color-difference map or a differential spectrum as the spectrum.

Where the variation in the optical signal is manifested as a change in optical absorption characteristics, irradiation of the gas-sensitive material with a light source of specific wavelengths (encompassing the ultraviolet to visible range) may result in the emergence of new absorption peaks, the disappearance of existing absorption peaks, or shifts in peak positions. Absorption spectral are acquired by means of a spectrometer, and the spectral images obtained before and after excitation are compared, thereby generating the differential spectrum to serve as the final spectrum.

In this way, at least two sensing units contain different gas-sensitive materials, thereby enabling selective detection of gases.

The specific implementation of the rapid sex identification method specifically includes the following steps:
(1) Selecting breeding eggs; subjecting the breeding eggs to cleaning, disinfection, and like treatments, and introducing the breeding eggs into incubation;

In step (1), the “breeding egg” is selected to be a poultry breeding egg, such as, by way of example and not limitation, a chicken egg, duck egg, or goose egg. The incubation conditions may be carried out in accordance with known standard incubation methods.
(2) Placing the optical sensor in a breeding egg environment to allow sufficient reaction with gases emitted therefrom; or, alternatively, collecting the gas from the breeding egg and exposing the collected gas to the optical sensor;

The reaction mode in step (2) includes contact and non-contact modes between the optical sensor and the breeding egg.

The contact mode includes, without limitation, spray-coating the gas-sensitive material onto the eggshell such that the gas-sensitive material directly reacts with volatile gases emitted by the breeding egg, or adhering a flexible sensor to the surface of the eggshell so that the flexible sensor reacts with volatile gases emitted by the breeding egg.

The non-contact mode includes, without limitation, placing the sensor and the breeding egg within the same chamber such that the gas-sensitive material does not physically contact the breeding egg but reacts with the gas volatilized or emitted therefrom; or using a suction pump or a gas sampling bag to first collect the gas volatilized or emitted from the breeding egg and subsequently delivering the collected gas to the optical sensor for reaction. Ultimately, this causes the gas-sensitive material to produce changes in optical signals.
(3) Acquiring, by an optical information acquisition system, optical images of the optical sensor before and after the step (2).

The optical information acquisition system in step (3) includes, without limitation, industrial camera acquisition systems, smartphones, scanners, microplate readers, fluorescence spectrophotometers, color-recognition devices, and ultraviolet spectrophotometers, for acquiring optical signals from sensors.

The obtained optical signal data includes but is not limited to RGB color information, ultraviolet spectral signals, fluorescence intensity signals, etc.
(4) Establishing a dataset from the collected optical images in the foregoing step;

The dataset in step (4) is composed of preprocessed color-difference maps and corresponding sex annotation labels, which are used for subsequent model training.
(5) Constructing a model;

In step (5), machine-learning techniques are employed, utilizing data analysis software including but not limited to Python and MATLAB, to establish a sex identification model for breeding eggs based on their optical signals.

In step (5), the breeding egg sex identification model for binary classification is established on the basis of collected optical signals, including images or spectra. Thereafter, under conditions in which the sex of the breeding eggs is known, the identification model is trained by means of transfer learning techniques to obtain the model parameters; upon completion of such training, the trained weights are saved so as to produce the breeding egg sex identification model. The model includes, without limitation, principal component analysis (PCA), convolutional neural networks (CNN), decision trees and random forests, support vector machines (SVM), artificial neural networks (ANN), and the like.
(6) Finally, the trained breeding egg sex identification model is used to process optical images acquired by the optical sensor after interaction with gases emitted from the breeding egg, and to output a determination result of the sex of the breeding egg, thereby ultimately achieving early-stage, non-destructive sex identification of breeding eggs.

Embodiments of the present disclosure are as follows:

### Embodiment 1

Polyethersulfone (PES) film is selected as the sensor substrate. The film is cut into rectangular substrates each measuring 5.0 mm×6.0 mm, which are uniformly arranged, in a planar array, within a polytetrafluoroethylene (PTFE) frame plate, thereby forming a film-array sensor.

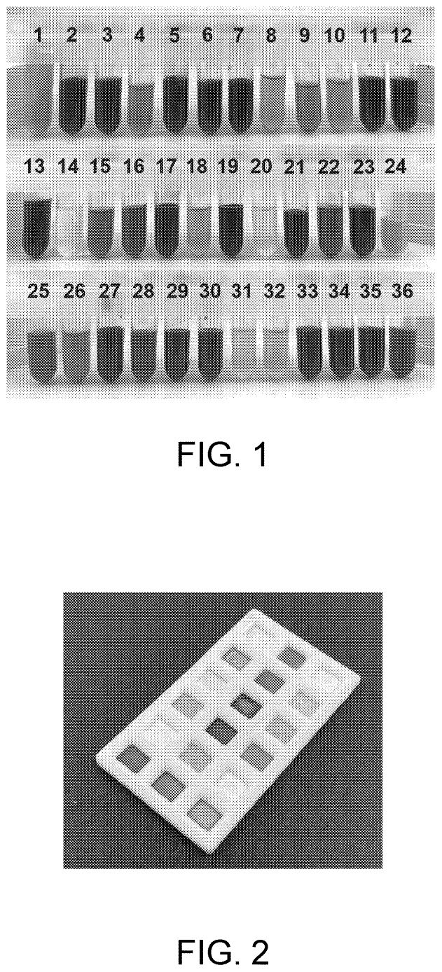

Four milligrams of each of thirty-six dyes are respectively weighed into thirty-six centrifuge tubes, and, upon the addition of 1 mL of ethanol and subsequent ultrasonication, dye solutions of various types are obtained as illustrated in FIG. 1. The thirty-six dyes, classified according to their chemical structures, include aniline dyes, phenolic dyes, azo dyes, aromatic amine dyes, ketone dyes, aldehyde dyes, pyrrole dyes, indole dyes, phthalocyanine dyes, anthraquinone dyes, nitrobenzene dyes, phenol-aldehyde dyes, benzoquinone dyes, carbazole dyes, pyrazole dyes, acid dyes, azo-ketone dyes, and copper phthalocyanine dyes, including, by way of example, bromocresol green, methyl red, N, N′-diphenyl-1,4-phenylenediamine, and toluidine blue.

Aliquots (4 μL each) of the thirty-six dye solutions obtained as described above are sequentially dispensed, using a micropipette, onto the PES film substrate. The substrate is then dried on a hot plate at 36 °C for 48 hours, thereby yielding the colorimetric sensing array shown in FIG. 2, wherein each circular spot formed by deposition of an individual dye solution constitutes the sensing unit.

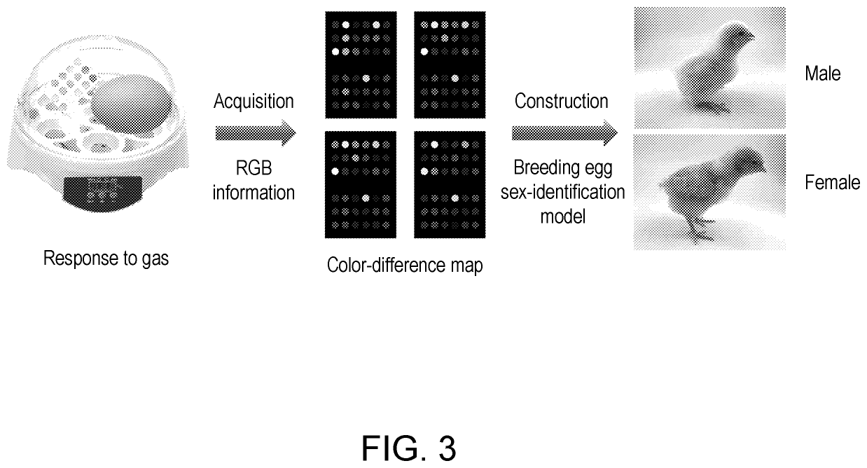

As depicted in FIG. 3, the detection procedure is as follows. Breeding eggs of Jinghong No. 1 chickens are selected as test specimens. The breeding eggs are wiped and disinfected with a 75% ethanol solution and allowed to air-dry. The eggs weigh between 49.82 g and 69.72 g, have a longitudinal axis of 53.13 mm to 61.81 mm and a transverse axis of 40.03 mm to 51.87 mm, and exhibit an egg shape index (longitudinal axis/transverse axis) of 1.10 to 1.45. Each breeding egg is placed in an individual incubator. The incubation conditions are a temperature of 37 °C to 38 °C and a relative humidity of 60% to 70%, with egg turning conducted at ninety-minute intervals.

First, the pre-reaction image data of a colorimetric sensor array are acquired by means of the industrial camera acquisition system. The camera focal length, aperture, and object distance are adjusted, and the exposure parameter in the industrial camera software is set to 120 ms. The image acquisition is conducted within a photographic dark box under incandescent-light conditions, with the light source being a 20 W to 40 W cool white LED having a correlated color temperature of 6000 K to 7000 K, thereby ensuring that the acquired image information is not affected by ambient illumination or other extraneous factors.

Thereafter, the colorimetric sensor is disposed together with a breeding egg at day 6 of the incubation period within an independent incubator at a location proximate to the air cell of the breeding egg, thereby establishing contact between the sensor and the gases generated during respiration of the breeding egg. The sensing unit undergoes a color change, and images after a 24-hour reaction period are again obtained by means of the industrial camera.

Python-based image processing algorithms are employed to preprocess and denoise the acquired images of the colorimetric sensor array, for the purpose of reducing computational load, enhancing operational efficiency, and improving computational accuracy, and to extract the mean values of the red, green, and blue (R, G, B) color channels. The specific method includes: performing perspective correction on an image file and resizing the image file to fixed dimensions; converting the image to grayscale; applying Gaussian filtering; conducting image binarization by means of the Otsu's thresholding method to extract image contours; ranking the contours by area; and approximating the largest contour to obtain a quadrilateral, which delineates the optical sensor region within the image. The extracted area is partitioned, in accordance with the position of the sensing unit, into a 6 -by-6 grid of sub-regions. For each sub-region, the mean values of R, G, and B color channels within a 12-by-20-pixel area are determined, thereby yielding a set of R, G, and B values representative of the color of the corresponding sensing unit region.

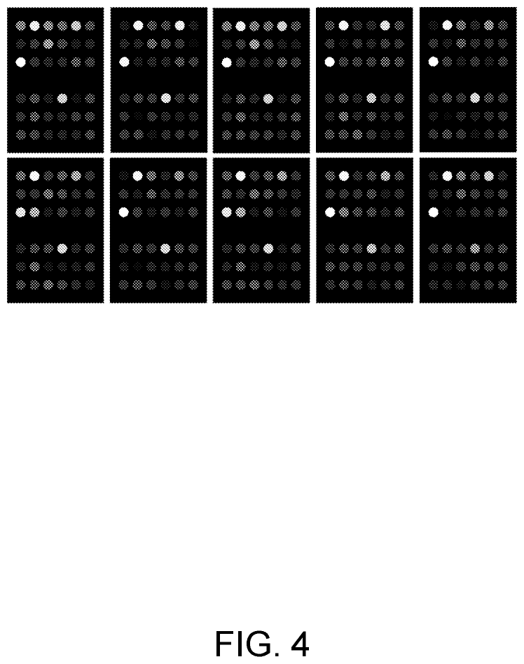

Image data obtained before and after the reaction (i.e., before and after placement in the vicinity of the breeding egg) are acquired and processed, and the differences in the R, G, and B values for each sensing unit are calculated. The differences in the R, G, and B values for each sensing unit are then amplified (converted from 4-19 to 0-255) and subsequently plotted to generate a color-difference map as illustrated in FIG. 4.

Upon the successful hatching of the chicks, the actual sex of the chickens is ascertained by methods including, without limitation, feather color assessment and cloacal examination, and the color difference maps are respectively defined as the color difference map for female chickens and the color difference map for male chickens.

The images are subjected to data augmentation, pursuant to which brightness, contrast, and saturation are, respectively and independently, randomly adjusted within a range of [−0.2, 0.2], and each image is, with a probability of 0.5, converted to a grayscale image.

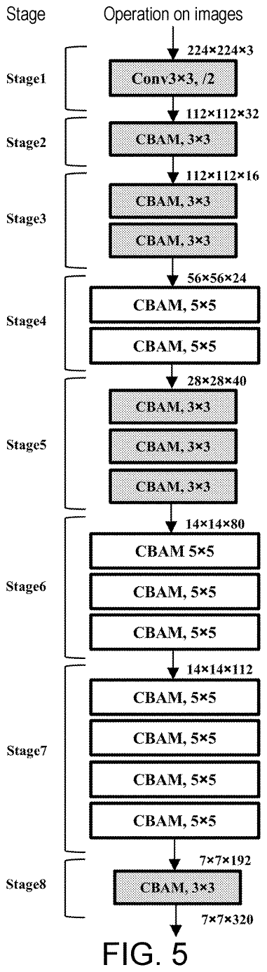

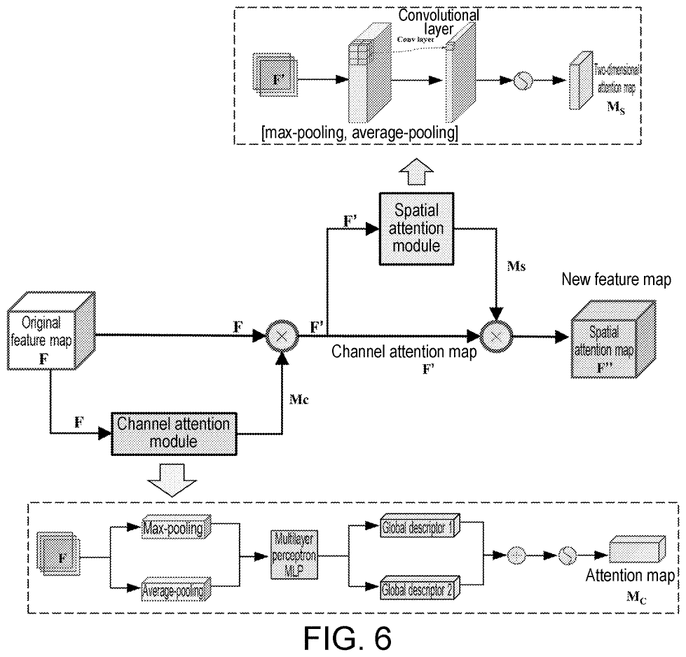

This disclosure sets forth the design and construction of an EfficientNet-B0-CBAM model for the rapid sex identification of breeding eggs, wherein the model adopts the EfficientNet-B0 baseline network as the backbone architecture and incorporates a Convolutional Block Attention Module (CBAM). The model thereby preserves the structural advantages of EfficientNet, which has achieved state-of-the-art accuracy on the ImageNet dataset, while effecting an order-of-magnitude reduction in the number of parameters relative to networks such as ResNet-50 and InceptionV2. The network architecture of EfficientNet-B0 is illustrated in FIG. 5. In the foregoing model, the CBAM is employed in lieu of the original Mobile Inverted Bottleneck Convolution (MBConv) modules and sequentially applies the Channel Attention Module (CAM) and the Spatial Attention Module (SAM). By assigning differential weights to information that is to be emphasized or suppressed, the module facilitates information flow within the network and yields improved performance in convolutional operations. The adopted CBAM structure is depicted in FIG. 6.

The breeding egg sex identification model is adopted as the EfficientNet-B0-CBAM model and is divided into nine stages performed sequentially:

The first stage is a backbone network, which employs only convolutional modules and, specifically, is constituted exclusively of convolutional modules. Each convolutional module is mainly formed, in sequence, by a 3×3 convolution operation with a stride of 2, a batch normalization layer, and a Swish activation function.

The second through eighth stages adopt CBAM network, each stage employing a feature-extraction module CBAM mainly composed of a CAM and a SAM. The second stage includes one feature-extraction module CBAM of size 3×3; the third stage includes two feature-extraction modules CBAM of size 3×3; the fourth stage includes two feature-extraction modules CBAM of size 5×5; the fifth stage includes three feature-extraction modules CBAM of size 3×3; the sixth stage includes three feature-extraction modules CBAM of size 5×5; the seventh stage includes four feature-extraction modules CBAM of size 5×5; and the eighth stage includes one feature-extraction module CBAM of size 3×3.

The feature-extraction module CBAM includes the CAM and the SAM, wherein an original feature map F input to the feature-extraction module CBAM is processed by the CAM to obtain a channel feature map Mc. The channel feature map Mc and the input original feature map F are then subjected to a multiplication to obtain a channel attention map F′. Specifically, the multiplication of Mc and F enables selective amplification or attenuation of channel features. The channel attention map F′ is subsequently processed by the SAM to obtain a spatial feature map Ms. Then, the spatial feature map Ms and the channel attention map F′ are subjected to the multiplication to obtain a spatial attention map F″, which serves as the output of the feature-extraction module CBAM.

The CAM includes a max-pooling layer, an average-pooling layer, and a multilayer perceptron MLP. The original feature map F is respectively processed by the max-pooling layer and the average-pooling layer and then input into the multilayer perceptron MLP to respectively obtain a first global descriptor (vector1) and a second global descriptor (vector2). The first global descriptor vector1 and the second global descriptor vector2 are summed and subsequently subjected to convolutional layer processing to obtain the channel feature map Mc. The two descriptors, respectively, capture the importance and the distributional characteristics of the distinct channels within the feature map. Thereafter, the descriptors are delivered to a series of convolutional layers, ultimately producing an attention map Mc having a channel count identical to that of the input feature map.

The SAM includes a max-pooling layer and an average-pooling layer. The channel attention map F′ is processed by the max-pooling layer and the average-pooling layer and the resulting outputs are concatenated, and the concatenated output is then subjected to convolutional layer to obtain the spatial feature map Ms. This procedure first computes, for each position within the feature map, a mean value AvgPool and a maximum value MaxPool. The foregoing two statistics are then concatenated and transmitted to a convolutional layer, thereby producing a single two-dimensional spatial feature map Ms. The attention map Ms is likewise multiplied by the feature map F′, thereby enabling the model to focus on salient regions and, through the convolutional layer, yielding the spatial attention map F″.

Finally, a ninth stage is formed by sequentially connecting, in series, a 1 ×1 convolution operation, a global average-pooling layer, a fully connected layer, and a Softmax activation function.

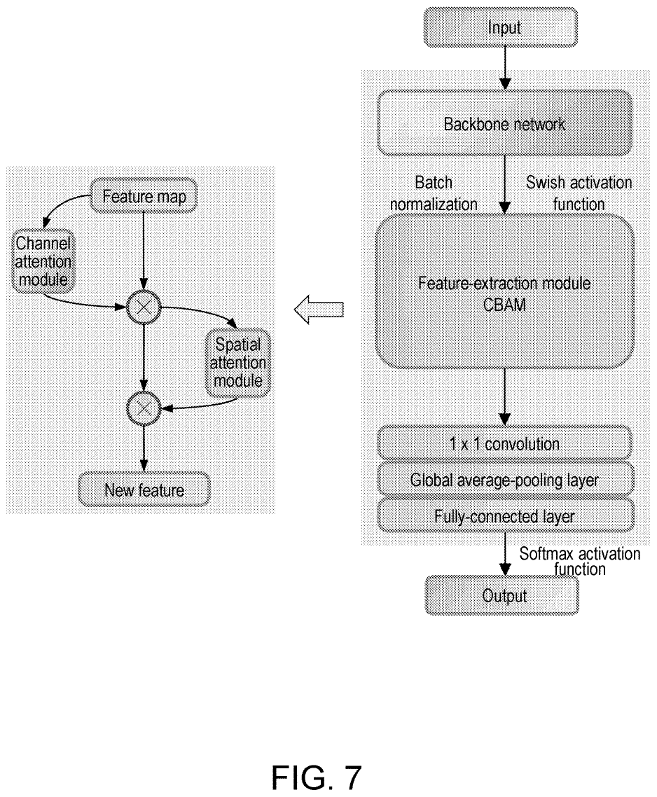

In the present embodiment, the first stage includes a 3×3 standard convolution with a stride of 2, followed by a Batch Normalization (BN) layer and a Swish activation function. The second through eighth stages employ the CBAM module in lieu of the original MBConv module, wherein, in each stage, the number of repetitions of the module corresponds to the layer count Li. The final stage includes a 1×1 convolution, global average pooling, and a fully connected layer, to which the Softmax activation function may be appended for classification. The overall network topology of the model is as illustrated in FIG. 7.

Training is conducted by means of transfer learning, whereby the ImageNet dataset is utilized for pre-training and the resulting parameters are thereafter transferred to a modified EfficientNet architecture for fine-tuning using a sample dataset. Upon completion of training, a breeding egg sex identification model is obtained. During the training process, an Adam optimizer is employed to iteratively update the neural network's weight parameters on the basis of the training data, and a Softmax classifier is utilized to output the probabilities that an image belongs to either the male or female breeding egg class. The loss function employed is categorical cross-entropy. Upon completion of training, the model's weight parameters are saved.

In particular, an evaluation of model performance may be conducted using the following metrics, including:

Accuracy=(TP+TN)/(TP+TN+FP+FN)×100%

Recall=TP/(TP+FN)×100%

Precision=TP/(TP+FP)×100%

F1-score=[(Precision⁻¹ +Recall⁻¹)/2]⁻¹ ×100%

AUC=[TP/2(TP+FN)+TN/2(TN+FP)]×100%

TP: True Positive, determined as a positive sample, and actually is a positive sample;

TN: True Negative, determined as a negative sample, and actually is a negative sample;

FP: False Positive, determined as a positive sample, but actually a negative sample;

FN: False Negative, determined as a negative sample, but actually is a positive sample.

Accuracy: The identification accuracy of the model with respect to the dataset, i.e., the proportion of correctly identified samples to the total number of samples.

Recall: Recall refers to the proportion of samples that are truly positive among all truly positive samples in the samples predicted as positive by the classifier.

Precision: Precision refers to the proportion of samples that are truly positive among all samples predicted as positive by the classifier, relative to the total number of samples predicted as positive.

F1 score: F1 score is an indicator used for comprehensively considering precision and recall, which is the harmonic mean of precision and recall.

Area Under the Curve (AUC): AUC is a comprehensive evaluation indicator related to the ROC curve, which may intuitively compare and evaluate the performance of classifiers. The closer the ROC curve is to the upper left corner, the larger the area under the curve, and the better the performance of the classifier.

A VGGNet-16 model, a classical architecture employed for feature extraction, is comprised primarily of uniform convolutional layers and pooling layers. By increasing the depth of the network, VGGNet-16 may effectively extract complex features and thereby enhance classification performance. Its topology consists of sixteen layers, including thirteen convolutional layers and three fully connected layers. The convolutional layers utilize 3×3 kernels and adopt a ReLU activation function; sequences of convolutional layers are followed by max-pooling layers, and, ultimately, fully connected layers are employed to perform classification.

The pre-defined color difference map dataset is partitioned into a training set constituting 70% and a test set constituting 30% for the purposes of training and evaluating the model, and the corresponding data loaders are established. During the training process, the number of iterations is set at 100, a learning rate is fixed at 0.001, and the batch size is set to 8.

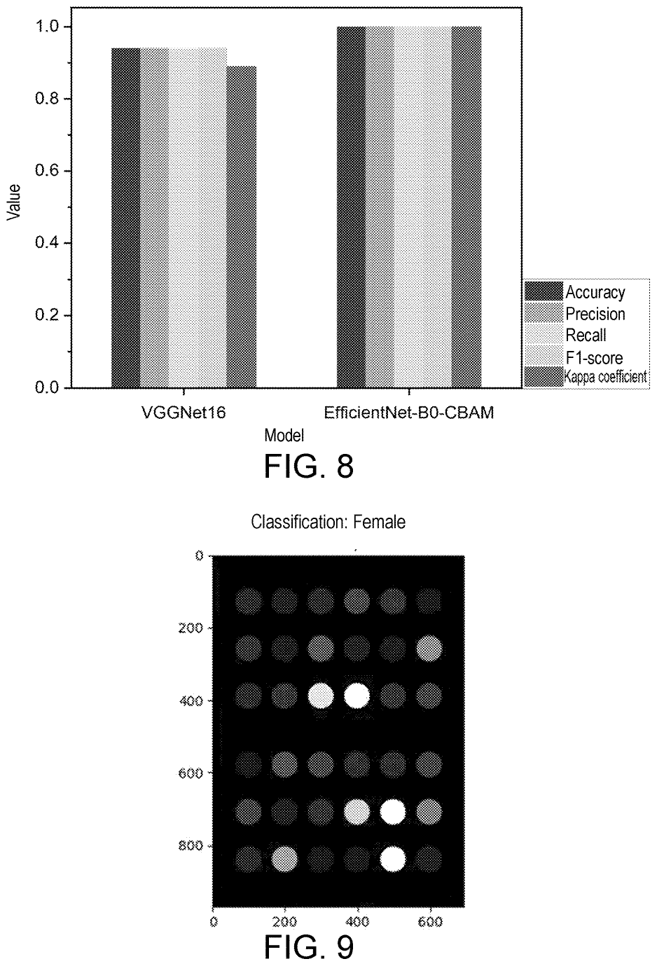

As shown in FIG. 8, the classical model VGGNet16 and the EfficientNet-B0-CBAM model of the present disclosure are respectively trained and tested on the breeding egg color difference map dataset. The results show that the classical VGGNet16 algorithm achieves an accuracy of 94.0%, whereas the breeding egg rapid sex identification algorithm EfficientNet-B0-CBAM proposed by the present disclosure achieves an accuracy of 100.0%, thereby improving the accuracy and efficiency of early sex identification of breeding eggs. Moreover, the precision, recall, and accuracy of EfficientNet-B0-CBAM are each higher than those of VGGNet16 in the comparative experiments.

The EfficientNet-B0-CBAM model that exhibits the highest accuracy in discriminating between female and male breeding eggs is employed for subsequent non-destructive sex identification of breeding eggs during the early incubation stage. As shown in FIG. 9, pursuant to the pre-established model, by inputting the colorimetric sensor color difference map obtained following its reaction with the breeding egg under test, the sex of the breeding egg is thereby successfully identified.

### Embodiment 2:

Eighteen dye powders are, respectively, weighed and placed upon a glass grinding plate; to each, gum arabic, ox gall, and glycerol are added dropwise in a mass ratio of 3:2:1:4. The resulting mixtures are ground with a muller for 15 minutes, thereby yielding distinct semi-fluid inks. The eighteen selected dyes, categorized by chemical structure, include indicator dyes, amine compounds, metal complexes, and other reagents, including but not limited to bromophenol red, bromocresol green, methyl red, diphenylamine, and a manganese porphyrin complex.

Breeding eggs of the Jinghong No. 1 chicken are selected as test specimens. Each breeding egg is surface-disinfected by wiping with a 75% ethanol solution and is then allowed to air-dry under ambient conditions. The eggs exhibit the following characteristics: a mass ranging from 49.82 g to 69.72 g; a longitudinal axis ranging from 53.13 mm to 61.81 mm; a transverse axis ranging from 40.03 mm to 51.87 mm; and an egg shape index (longitudinal axis/transverse axis) of 1.10 to 1.45.

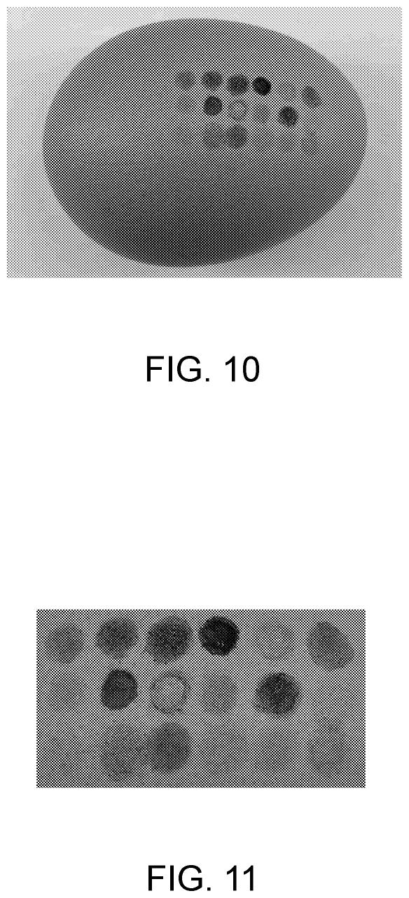

A transfer-printing array stamp including a 3×6 array is fabricated from a fibrous material, the raised portions of the stamp being circular and each measuring 3×3 mm. Using a micropipette, the eighteen dye inks obtained are sequentially applied to each circular raised portion of the transfer-printing array stamp. Thereafter, the ink-saturated stamp is promptly pressed onto the central region of a clean eggshell and allowed to air-dry at room temperature for 3 hours, thereby yielding a breeding egg bearing a tattoo sensor as shown in FIG. 10. Each circular dot formed on the breeding egg by each respective dye ink constitutes a sensing unit.

The pre-reaction image data of the tattooed breeding egg is collected from the sensor by means of the industrial camera acquisition system. The camera focal length, aperture, and object distance are adjusted, and the exposure parameter in the industrial camera software is set to 120 ms. The image acquisition is conducted within a photographic dark box under controlled lighting conditions, with the light source being a 20 W to 40 W cool white LED having a correlated color temperature of 6000 K to 7000 K, thereby ensuring that the acquired image information is not affected by ambient illumination or other extraneous factors.

Each tattooed breeding egg is placed in an individual incubator. The incubation conditions are a temperature of 37 °C to 38 °C and a relative humidity of 60% to 70%, with egg turning conducted at ninety-minute intervals, thereby ensuring stable interaction between the tattoo sensor and the gases generated during respiration of the breeding egg. The sensing unit undergoes a color change, and images of the tattooed breeding eggs after a consecutive 3-day reaction period are again obtained by means of the industrial camera.

Python-based image processing algorithms are employed to preprocess and denoise the acquired images of the tattoo sensor array, for the purpose of reducing computational load, enhancing operational efficiency, and improving computational accuracy, and to extract features from the red, green, and blue (R, G, B) channels of the images. The specific method includes: performing perspective correction on the curvature distortion present in the acquired breeding egg images, using distortion coefficients k1=0.1, k2=0.02, and k3=0.003. Thereafter, mapping calculations are performed to ensure that a center point (cx, cy) is correctly computed and that the mapped image exhibits appropriate distortion correction in the vicinity of the center point. Finally, image slicing is performed to ensure that the output image size is identical to that of the input image, so as to avoid blank areas at the boundaries. The output is the corrected tattoo breeding egg image as depicted in FIG. 11.

Each rectified image file is resized to fixed dimensions, and the distance from a left edge of the frame to the center point of a first circular dye-ink mark is identical across all files. Afterwards, the image is converted to grayscale and subjected to Gaussian filtering. Image binarization is conducted by means of the Otsu's thresholding method to extract image contours. The contours are ranked by contour area. The largest contour is approximated to obtain a quadrilateral, which delineates the optical sensor region within the image. The extracted area is partitioned, in accordance with the position of the sensing unit, into a 3-by-6 grid of sub-regions. For each sub-region, the mean values of the R, G, and B color channels within a 12-pixel area are obtained, thereby yielding a set of R, G, and B values representative of the color of the corresponding region.

Image data obtained before and after the reaction is acquired and processed, and the differences in the R, G, and B values for each sensing unit are calculated. The differences in the R, G, and B values for each sensing unit are then amplified (converted from 4-19 to 0-255) and subsequently plotted to generate the color-difference map.

Upon the successful hatching of the chicks, the actual sex of the chickens is ascertained by methods including, without limitation, feather color assessment and cloacal examination, and the color difference maps are respectively defined as the color difference map for female chickens and the color difference map for male chickens.

The images are subjected to data augmentation, pursuant to which brightness, contrast, and saturation are, respectively and independently, randomly adjusted within a range of [−0.2, 0.2], and each image is, with a probability of 0.5, converted to a grayscale image.

This disclosure sets forth the design and construction of the EfficientNet-B0-CBAM model for the rapid sex identification of breeding eggs, wherein the model adopts the EfficientNet-B0 baseline network as the backbone architecture and incorporates the CBAM module, in which the CAM and SAM are sequentially applied. By assigning differential weights to information that is to be emphasized or suppressed, the module facilitates information flow within the network, thereby enabling the extraction of higher-level feature representations during convolution operations. The feature map output after the image is processed by the CBAM module is classified by the fully connected layers and activation functions of the classification network, so as to output the sex identification result corresponding to the breeding-egg sample image. Details are set forth in Embodiment 1.

The classical VGGNet16 model is constructed; details are set forth in Embodiment 1.

The pre-defined color difference map dataset is partitioned into a training set constituting 70% and a test set constituting 30% for the purposes of training and evaluating the model, and the corresponding data loaders are established. During the training process, the number of iterations is set to 100, the learning rate is fixed at 0.001, and the batch size is set to 8.

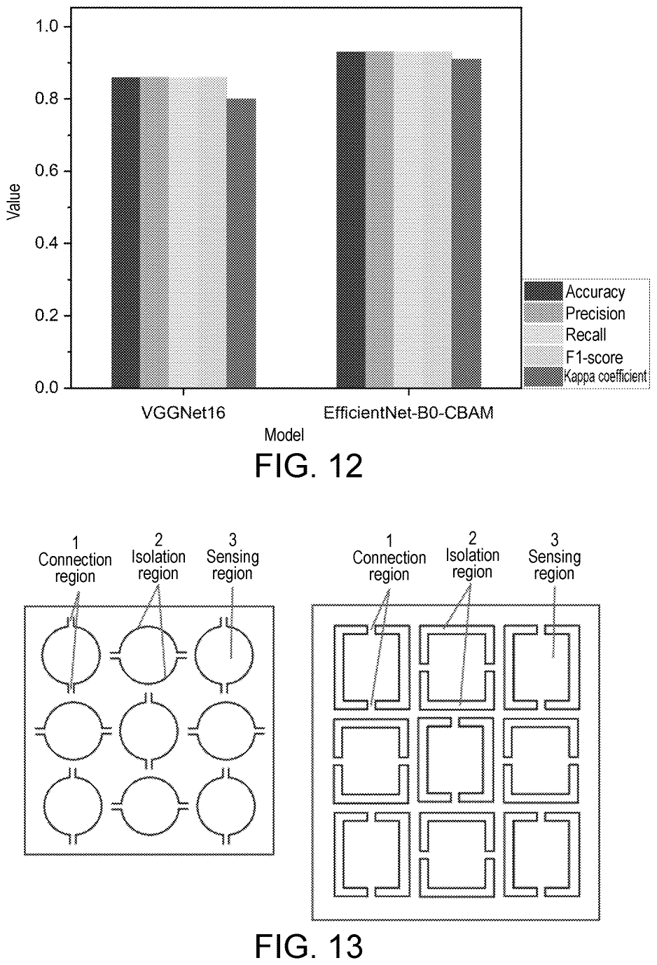

Using the classical model VGGNet16 and the EfficientNet-B0-CBAM model of the present disclosure, the breeding egg color difference map dataset is respectively subjected to training and testing. As shown in FIG. 12, the rapid sex identification algorithm for breeding eggs proposed herein, EfficientNet-B0-CBAM, achieves an accuracy of 93.0%, whereas the classical VGGNet16 model achieves an accuracy of 86.0%. The present disclosure thereby enhances the accuracy and efficiency of early sex identification of breeding eggs, and the precision, recall, and accuracy of EfficientNet-B0-CBAM each exceed those of VGGNet16 in the comparative experiments. Accordingly, EfficientNet-B0-CBAM is selected as the optimal model for subsequent non-destructive prediction of the sex of breeding eggs during the early incubation period.

### Embodiment 3

The PES film substrate is cut by means of laser cutting. First, an engraving path and parameters are designed using computer-aided design (CAD) software. The PES paper-based material is then placed on a worktable of a laser engraving system, and the system is configured in accordance with the designed engraving parameters, specifically: a vector mode is selected; laser power 5.0%; engraving speed is set to 45%; and PPI is set to 750. This process yields a rectangular sensor substrate measuring 27.6 mm×30.8 mm, including a uniformly distributed 3×3 array of nine sensing units, each individual sensing unit being rectangular and measuring 2.9 mm ×3.5 mm. Under the same operating conditions, a square sensor substrate measuring 13 mm×13 mm may be obtained, including nine uniformly distributed circular sensing units, each having a diameter of 3 mm. As shown in FIG. 13, the design of the sensor substrate includes a sensing region, a connection region, and an isolation region.

Nine types of materials possessing fluorescent properties are respectively weighed, 4 mg each, into nine centrifuge tubes. By adding 1 mL of ethanol to each tube and subjecting the mixtures to ultrasonic treatment, nine solutions or suspensions are obtained. The nine fluorescent sensing substances include amine dyes, phthalocyanine dyes, aldehyde dyes, and dyes of the nitrate class, including, by way of example, Lissamine Green, diphenylamine, and thymol blue.

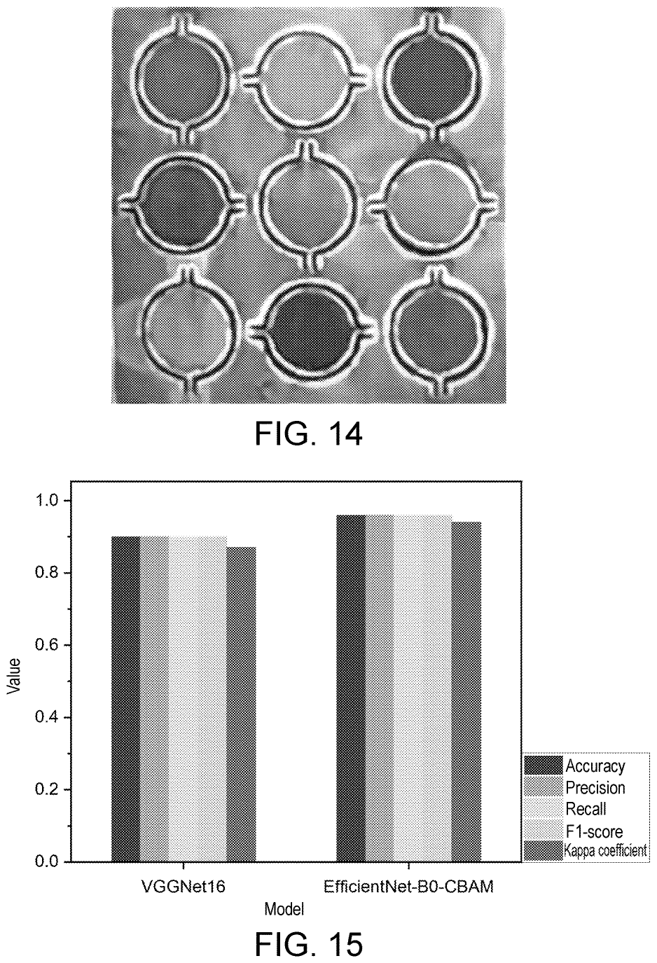

The nine resultant fluorescent sensing solutions are each aliquoted at 4 μL using a micropipette and, in sequence, dispensed dropwise onto the sensing regions of the sensor substrate. The deposits are then dried and allowed to stabilize for 48 hours on a heating plate maintained at 36 °C. Upon ultraviolet excitation at 365 nm, a fluorescent sensing array is obtained as shown in FIG. 14 (for illustrative purposes, the figure depicts circular sensing units).

Breeding eggs of Jinghong No. 1 chicken are selected as test specimens. The breeding eggs are wiped and disinfected with a 75% ethanol solution and allowed to air-dry. The eggs weigh between 49.82 g and 69.72 g, have a longitudinal axis of 53.13 mm to 61.81 mm and a transverse axis of 40.03 mm to 51.87 mm, and exhibit an egg shape index (longitudinal axis/transverse axis) of 1.10 to 1.45. Each breeding egg is placed in an individual incubator. The incubation conditions are a temperature of 37 °C to 38 °C and a relative humidity of 60% to 70%, with egg turning conducted at ninety-minute intervals.

The pre-reaction image data of the fluorescent sensor array are acquired by means of the industrial camera acquisition system. The camera focal length, aperture, and object distance are adjusted, and the exposure parameter in the industrial camera software is set to 120 ms. The image acquisition is conducted within a photographic dark box equipped with a 365-nm ultraviolet excitation light source, thereby ensuring that the acquired image information is not affected by ambient illumination or other extraneous factors.

Thereafter, the fluorescent sensor is disposed together with a breeding egg at day 5 of the incubation period within an independent incubator at a location proximate to the air cell of the breeding egg, thereby ensuring stable interaction between the sensor and the gases generated during respiration of the breeding egg. The sensing unit undergoes a color change, and images after a 24-hour reaction period are obtained by means of the industrial camera.

Python-based image processing algorithms are employed to preprocess and denoise the acquired images of the fluorescent sensor array, for the purpose of reducing computational load, enhancing operational efficiency, and improving computational accuracy, and to extract mean values of the red, green, and blue (R, G, B) color channels. The specific method includes: performing perspective correction on the image file and resizing the image file to fixed sizes; converting the image to grayscale; applying Gaussian filtering; conducting image binarization by means of the Otsu's thresholding method to extract image contours; ranking the contours by area; and approximating the largest contour to obtain a quadrilateral, which delineates the optical sensor region within the image. The extracted area is partitioned, in accordance with the position of the sensing unit, into a 3 -by-3 grid of sub-regions. For each sub-region, the mean values of R, G, and B color within a 12-by-12-pixel area are obtained, thereby yielding a set of R, G, and B values representative of the color of the corresponding sensing unit region.

Image data obtained before and after the reaction is acquired and processed, and the differences in the R, G, and B values for each sensing unit are calculated. The differences in the R, G, and B values for each sensing unit are then amplified (converted from 4-19 to 0-255) and subsequently plotted to generate the color-difference map.

Upon the successful hatching of the chicks, the actual sex of the chickens is ascertained by methods including, without limitation, feather color assessment and cloacal examination, and the color difference maps are respectively defined as the color difference map for female chickens and the color difference map for male chickens.

The images are subjected to data augmentation, pursuant to which brightness, contrast, and saturation are, respectively and independently, randomly adjusted within a range of [−0.2, 0.2], and each image is, with a probability of 0.5, converted to a grayscale image.

This disclosure sets forth the design and construction of the EfficientNet-B0-CBAM model for the rapid sex identification of breeding eggs, wherein the model adopts the EfficientNet-B0 baseline network as the backbone architecture and incorporates the CBAM module, in which the CAM and the SAM are sequentially applied. By assigning differential weights to information that is to be emphasized or suppressed, the module facilitates information flow within the network, thereby enabling the extraction of higher-level feature representations during convolution operations. The feature map output after the image is processed by the CBAM module is classified by the fully connected layers and activation functions of the classification network, so as to output the sex identification result corresponding to the breeding-egg sample image. Details are set forth in Embodiment 1.

The classical VGGNet16 model is constructed; details are set forth in Embodiment 1.

The pre-defined color difference map dataset is partitioned into a training set constituting 70% and a test set constituting 30% for the purposes of training and evaluating the model, and the corresponding data loaders are established. During the training process, the number of iterations is set to 100, the learning rate is fixed at 0.001, and the batch size is set to 8.

The classical model VGGNet16 and the EfficientNet-B0-CBAM model of the present disclosure are respectively trained and tested on the breeding egg color difference map dataset. The results in FIG. 15 show that the breeding egg rapid sex identification algorithm EfficientNet-B0-CBAM proposed by the present disclosure achieves an accuracy of 96.0%, whereas the classical model VGGNet 16 algorithm achieves an accuracy of 90.0%, the present disclosure improves the precision and efficiency of early sex identification of breeding eggs. Moreover, the precision, recall, and accuracy of EfficientNet-B0-CBAM are each higher than those of VGGNet16 in the comparative experiments. EfficientNet-B0-CBAM is hereby selected and designated as the optimal model for use in the subsequent non-destructive prediction of the sex of breeding eggs during the early stage of incubation.

The foregoing embodiments are provided solely for purposes of illustrating the present disclosure and are not intended to limit the technical solutions described herein; accordingly, although the disclosure has been described in detail with reference to the foregoing embodiments, those of ordinary skill in the art will understand that modifications, variations, and equivalent substitutions may be made to the disclosure, and any and all technical solutions and improvements that do not depart from the spirit and scope of the disclosure are intended to be encompassed within the scope of protection defined by the appended claims.

What is claimed is:

1. A method for rapid identification of breeding egg sex utilizing the optical sensor,
wherein the optical sensor comprises a substrate and at least two sensing units loaded on the substrate, the sensing units comprising gas-sensitive materials capable of reacting with gas and inducing optical signal changes;
the gas-sensitive material is provided for adsorbing the gas or reacting with the gas, causing changes in optical properties of the material, including changes in light absorption/reflection characteristics, photoluminescence changes, and color changes, thereby detecting gas composition and concentration;
or wherein the gas-sensitive material comprises, but is not limited to, chromogenic moieties that undergo a colorimetric change in response to the gas, comprising dyes, nanoporous materials, and gold nanoparticles, or fluorescent and/or phosphorescent moieties that exhibit a luminescence response to the gas, comprising metal-organic frameworks (MOFs), quantum dots, and carbon dots,
wherein the method adopts the optical sensor composed of the plurality of sensing units placed in an environment near the breeding egg to react, or uses the sensor to react with a gas from the collected breeding egg, and then collects the optical signal changes of each of the sensing units before and after placement for processing and analysis to obtain the sex of the breeding egg.

2. The method for rapid identification of breeding egg sex utilizing the optical sensor according to claim 1, wherein the optical sensor comprises the sensing units containing at least two different gas-sensitive materials, and the method collects the optical signal changes of the sensing units with the different gas-sensitive materials and combines them to form a specific spectrum, analyzes and processes the spectrum, and then identify the sex of the breeding egg.

3. The method for rapid identification of breeding egg sex utilizing the optical sensor according to claim 2, wherein the plurality of sensing units with the different gas-sensitive materials are arranged on the same substrate, and visual images or spectral images are collected under both conditions before and after the entire substrate is placed near the breeding egg, and a difference map or a color-difference map of the visual images or the spectral images under conditions before and after the placement near the breeding egg is taken as the spectrum.

4. The method for rapid identification of breeding egg sex utilizing the optical sensor according to claim 3, wherein when the optical signal change is a color change, natural light or an artificial light source illuminates the gas-sensitive material, and the color change of reflected light is collected by cameras or camcorders, the color-difference map is formed as the spectrum by comparing the images collected before and after;
when the optical signal change is a photoluminescence change, a light source with a specific fixed wavelength is adopted as excitation light to irradiate the gas-sensitive material, which causes the material to emit fluorescence or phosphorescence by affecting a luminous efficiency, a luminous intensity, or a luminous wavelength of the material, and emission spectrum data or images are collected by a spectrometer or a camera, spectral image differences before and after excitation are compared to generate the color-difference map or a differential spectrum as the spectrum;
when the optical signal change is a light absorption characteristic change, the gas-sensitive material is illuminated through a specific fixed wavelength light source, causing the material to exhibit new absorption peaks, disappearance of original absorption peaks, or peak position shifts, a spectrometer is utilized to collect absorption spectral, and the spectral image differences before and after excitation are compared, thereby generating the differential spectrum as the final spectrum.

5. The method for rapid identification of breeding egg sex utilizing the optical sensor according to claim 3, wherein the collected images are processed in the following manner to obtain spectra:
first, a perspective transformation is performed on an image by mapping four designated points in the image—namely, upper-left, upper-right, lower-right, and lower-left—to a standardized rectangle, thereby effecting perspective correction and obtaining a target image;
thereafter, image preprocessing is performed to adjust sizes of the image, convert the image to grayscale, and subsequently apply Gaussian filtering to reduce noise;
thereafter, binarization is performed on the image using Otsu's thresholding method to extract contours of the image, wherein the contours are ranked by contour area, a contour of a greatest area is selected to be subjected to an approximation operation to obtain a quadrilateral, the quadrilateral represents an optical sensor region within the image;
subsequently, after the detected contour region is converted into a rectified frontal view by means of the perspective transformation, rectangular markers are drawn in accordance with locations of the sensing units, and a plurality of regions of interest are cropped, each of the regions of interest comprising one and only one sensing unit;
finally, mean values of R, G, and B color within the region of interest are calculated and designated as R, G, and B values of the sensing unit corresponding to the region, on basis of the R, G, and B values of the sensing unit, the color-difference map/the differential spectrum is generated, which serves as the spectrum.

6. The method for rapid identification of breeding egg sex utilizing the optical sensor according to claim 1, wherein the method specifically comprises the following steps:
(1) selecting the breeding eggs, subjecting the breeding eggs to cleaning, disinfection, and like treatments, and introducing the breeding eggs into incubation;
(2) placing the optical sensor in a breeding egg environment to allow sufficient reaction with the gas from the breeding egg; or, alternatively, collecting the gas from the breeding egg and exposing the collected gas to the optical sensor;
(3) acquiring, by an optical information acquisition system, optical signals of the optical sensor before and after the step (2);
(4) data processing and model construction: performing data preprocessing and feature extraction on the acquired optical signals and constructing a breeding egg sex-identification model, and thereafter training the constructed breeding egg sex identification model under known conditions;
(5) finally, using the trained breeding egg sex identification model to process the acquired optical signals, in a scenario under test and an environment where the breeding egg is present, by the optical sensor, thereby obtaining an identification result of the sex of the breeding egg.

7. The method for rapid identification of breeding egg sex utilizing the optical sensor according to claim 6, wherein the reaction mode in the step (2) comprises contact and non-contact modes between the optical sensor and the breeding egg;
the contact mode comprises, but is not limited to, spray-coating the gas-sensitive material onto eggshell such that the gas-sensitive material directly reacts with volatile gases emitted by the breeding egg, or adhering a flexible sensor to a surface of the eggshell so that the flexible sensor reacts with the volatile gases emitted by the breeding egg;
the non-contact mode comprises, but is not limited to placing the sensor and the breeding egg within the same chamber such that the gas-sensitive material does not physically contact the breeding egg but reacts with the gas volatilized or emitted therefrom, or using a suction pump or a gas sampling bag to first collect the gas volatilized or emitted from the breeding egg and subsequently deliver the collected gas to the optical sensor for reaction.

8. The method for rapid identification of breeding egg sex utilizing the optical sensor according to claim 6, wherein in the step (3), the optical information acquisition system comprises, but is not limited to, industrial camera acquisition systems, smartphones, scanners, microplate readers, fluorescence spectrophotometers, color-recognition devices, and ultraviolet spectrophotometers, for acquiring optical and/or visual signal data from the sensor.

9. The method for rapid identification of breeding egg sex utilizing the optical sensor according to claim 6, wherein in the step (4), the breeding egg sex identification model for binary classification is established on basis of the acquired optical signals, comprising images or spectra, wherein the breeding egg sex identification model comprises, but is not limited to, principal component analysis (PCA), convolutional neural networks (CNN), decision trees and random forests, support vector machines (SVM), artificial neural networks (ANN), and the like.

10. The method for rapid identification of breeding egg sex utilizing the optical sensor according to claim 6, wherein the breeding egg sex identification model is divided into nine sequential stages,
the first stage is a backbone network, which employs only convolutional modules, each of the convolutional modules is formed, in sequence, by a 3×3 convolution operation with a stride of 2, a batch normalization layer, and a Swish activation function;
the second through the eighth stages adopt a CBAM network, each of the stages employing a feature-extraction module CBAM mainly composed of a channel attention module CAM and a spatial attention module SAM, the second stage comprises one feature-extraction module CBAM of size 3×3, the third stage comprises two feature-extraction modules CBAM of size 3×3, the fourth stage comprises two feature-extraction modules CBAM of size 5×5, the fifth stage comprises three feature-extraction modules CBAM of size 3×3, the sixth stage comprises three feature-extraction modules CBAM of size 5×5, the seventh stage comprises four feature-extraction modules CBAM of size 5×5, and the eighth stage comprises one feature-extraction module CBAM of size 3×3;
the feature-extraction module CBAM comprises the channel attention module CAM and the spatial attention module SAM, wherein an original feature map F input to the feature-extraction module CBAM is processed by the channel attention module CAM to obtain a channel feature map Mc, the channel feature map Mc and the input original feature map F are then subjected to a multiplication to obtain a channel attention map F′, the channel attention map F′ is subsequently processed by the spatial attention module SAM to obtain a spatial feature map Ms, then, the spatial feature map Ms and the channel attention map F′ are subjected to the multiplication to obtain a spatial attention map F″, which serves as an output of the feature-extraction module CBAM;
the channel attention module CAM comprises a max-pooling layer, an average-pooling layer, and a multilayer perceptron MLP, the original feature map F is respectively processed by the max-pooling layer and the average-pooling layer and then input into the multilayer perceptron MLP to respectively obtain a first global descriptor vector1 and a second global descriptor vector2, the first global descriptor vector1 and the second global descriptor vector2 are summed and subsequently subjected to a convolutional layer processing to obtain the channel feature map Mc;
the spatial attention module SAM comprises a max-pooling layer and an average-pooling layer, the channel attention map F′ is processed by the max-pooling layer and the average-pooling layer and is concatenated, and the concatenated output is then subjected to the convolutional layer processing to obtain the spatial feature map Ms;
finally, the ninth stage is formed by sequentially connecting a 1×1 convolution operation, a global average-pooling layer, a fully connected layer, and a Softmax activation function.
